(19) United States
(12) Patent Application Publication
(10) Pub. No.: US 2021/0338994 A1
(43) Pub. Date: Nov. 4, 2021

(54) METHOD AND APPARATUS FOR TREATING TENSION PNEUMOTHORAX USING A RAPID DEPLOYMENT CHEST PORT

(71) Applicant: Quick Tube Medical, LLC, Knoxville, IN (US)

(72) Inventors: Allen Carmichael SMITH, Lebanon, TN (US); Ryan LUTTRELL, Lebanon, TN (US); Byron SMITH, Nashville, TN (US); Christen SPRINGS, Houston, TX (US)

(73) Assignee: Quick Tube Medical, LLC, Knoxville, TN (US)

(21) Appl. No.: 17/314,004

(22) Filed: May 6, 2021

Related U.S. Application Data
(63) Continuation of application No. PCT/US2019/063848, filed on Nov. 29, 2019.
(60) Provisional application No. 62/773,765, filed on Nov. 30, 2018.

(51) Int. Cl. A61M 39/02

(52) U.S. Cl. CPC A61M 39/0247; A61M 2039/0205; A61M 2210/101; A61M 2039/027; A61M 2039/0276; A61M 2039/0252

(57) ABSTRACT

The present disclosure provides apparatus and method for treating tension pneumothorax by using a rapid deployment chest port. The rapid deployment chest port can penetrate a patient's body to access a distressed pleural space. The rapid deployment chest port may create an airtight seal between the inside and outside of the patient's body and, when expanded, allow air or fluid to flow in one direction from inside the body to outside the body.

### CROSS-REFERENCE TO RELATED PATENT APPLICATION

This patent application is a Continuation of International Patent Application No. PCT/US2019/063848 filed Nov. 29, 2019, which claims the benefit under 35 U.S.C. § 119(e) of U.S. patent application Ser. No. 62/773,765, entitled “METHOD AND APPARATUS FOR TREATING TENSION PNEUMOTHORAX USING A RAPID DEPLOYMENT CHEST PORT,” filed on Nov. 30, 2018, both of which are incorporated herein by reference in their entirety.

### FIELD

The present disclosure relates to a method and apparatus for treating tension pneumothorax.

### BACKGROUND

Tension pneumothorax is the progressive build-up of air within the pleural space, usually due to a lung laceration which allows air to escape into the pleural space but not to return. In effect, this creates a one-way valve through which air or fluid escapes the lungs.

Progressive build-up of pressure in the pleural space pushes the mediastinum to the opposite hemithorax, and obstructs venous return to the heart. This leads to circulatory instability and may result in traumatic arrest.

Currently, the most effective treatment for tension pneumothorax is chest tube placement. Once a chest tube is inserted into the pleural space, usually through blunt dissection, the tension is decompressed. However, this takes time that the patient may not have and risks complications, including requiring suturing to secure the chest tube to the patient to reduce migration of the tube and the potential for creating inconsistent incision sizes that may lead to infection and/or requiring suturing.

### SUMMARY

Accordingly, the present invention provides a method and apparatus for accessing a patient's pleural space using a rapid deployment chest port.

In an aspect, the rapid deployment chest port includes a frame, a plunger with a blade at the distal end, where the plunger is contained within a lumen of the frame, and a stabilizing component configured to stabilize the frame inside and outside the chest cavity of a patient.

In another aspect, the stabilizing component includes an internal expanding flange attached to the outer diameter of the frame; and an insertion stabilization platform attached to the outer diameter of the frame proximal to the internal expanding flange.

Also provided herein is a method of removing air or fluid contained within a pleural space of a mammalian patient. In an aspect, the method may include inserting the blade of the plunger and distal portion of the frame of the rapid deployment chest port into a patient's chest cavity, expanding the stabilizing component to expand in at least the inside of the patient's chest cavity; and removing the plunger from the plunger port.

In another aspect, the stabilization component includes an internal expanding flange attached to the outer diameter of the frame, and an insertion stabilization platform attached to the outer diameter of the frame proximal to the internal expanding flange, such that the method includes expanding the internal expanding flange.

Additional aspects and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates an exemplary variation of a rapid deployment chest port in a contracted state.

FIG. 2 illustrates an exemplary variation of a rapid deployment chest port in an expanded state.

FIG. 3 illustrates an exemplary variation of the method for using a rapid deployment chest port.

FIG. 4 illustrates alternative views of an exemplary variation of a rapid deployment chest port.

FIG. 5 illustrates additional alternative views of an exemplary variation of a rapid deployment chest port.

FIG. 6 illustrates additional alternative views of an exemplary variation of a rapid deployment chest port.

FIG. 7 illustrates additional alternative views of an exemplary variation of a rapid deployment chest port.

FIG. 8A illustrates an exemplary variation the distal end of the rapid deployment chest port.

FIG. 8B illustrates an exemplary variation the distal end of the rapid deployment chest port.

FIG. 9A illustrates an exemplary variation of an insertion stabilization platform with a fixation flexure.

FIG. 9B illustrates the distal end of the rapid deployment chest port with an exemplary variation of the insertion stabilization platform with a fixation flexure.

FIG. 10A illustrates an exemplary variation of a compression fitting based insertion stabilization platform.

FIG. 10B illustrates an exemplary variation of the compression fitting based insertion stabilization platform.

FIG. 11A illustrates an internal expanding flange as a balloon, as described in various illustrative embodiments.

FIG. 11B illustrates an internal expanding flange as an expandable nitinol ascot, as described in various illustrative embodiments.

FIG. 12 illustrates additional alternative views of an exemplary variation of a rapid deployment chest port.

FIG. 13 illustrates an exemplary variation the distal end of the rapid deployment chest port.

FIG. 14 illustrates an exemplary variation of the method for treating tension pneumothorax using a rapid deployment chest port.

### DETAILED DESCRIPTION

In the following sections, detailed descriptions of examples and methods of the disclosure will be given. The description of both preferred and alternative examples are exemplary only, and it is understood that to those skilled in the art that variations, modifications, and alterations may be apparent. It is therefore to be understood that the examples do not limit the broadness of the aspects of the underlying disclosure as defined by the claims.

For purposes of this description, “distal” refers to the end extending into a body and “proximal” refers to the end extending out of the body.

For purposes of this description “connected to” includes two components being directly connected or indirectly connected with intervening components.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term.

The present disclosure provides generally for methods and an apparatus for treating tension pneumothorax using a rapid deployment chest port. According to the present disclosure, a rapid deployment chest port is inserted into the pleural area of a patient's body using a sharpened surface, such as a blade, needle, sharp tip, or knife edge. The sharpened surface may be attached to the rapid deployment chest port. Following insertion, the rapid deployment chest port may be expanded to open a cavity to relieve pressure from air and/or fluid buildup within the pleural space. In some variations, the rapid deployment chest port may further use suction to remove fluid from the pleural space.

The rapid deployment chest port allows for quick, standardized insertion of a chest tube without requiring creating an incision with a scalpel prior to insertion, as is current practice. Making an incision with a scalpel leads to inconsistent incisions that may be too large for the chest port, such that there may be an open wound around the chest port that may require suturing. Thus, the rapid deployment chest port provides less risk for infection in the patient because it creates a standardized incision that is the exact size needed for the rapid deployment chest port. In addition, a standard chest port requires suturing to stabilize the chest port so that it does not migrate within the patient. This requires additional time in the placement of the chest tube before the patient may be treated. The separate incision and suturing may lead to standard chest tubes taking several minutes to be inserted and ready for use. Because the rapid deployment chest port does not require a separate incision or any additional suturing, it provides for a reduction in the amount of time to insert the chest port and begin treating the patient. In some variations, the rapid deployment chest port may be deployed within 20 seconds. In some variations, the rapid deployment chest port may be deployed within 30 seconds. In some variations, the rapid deployment chest port may be deployed within 60 seconds. In some variations, the rapid deployment chest port may be deployed within 90 seconds.

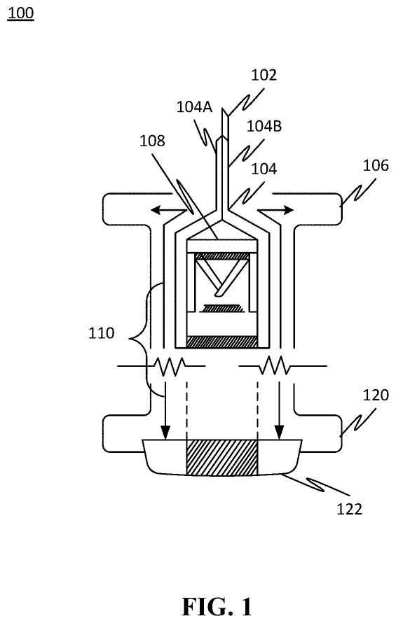

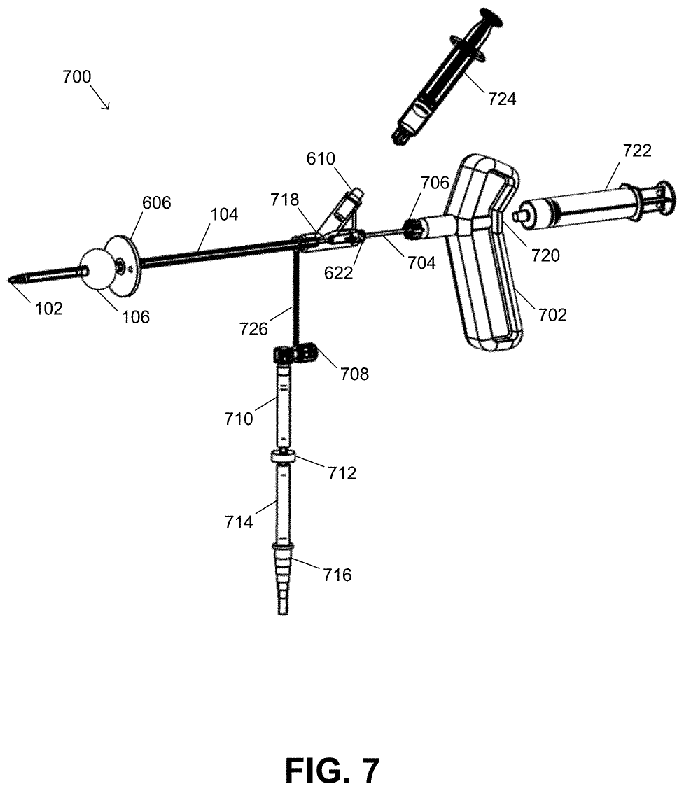

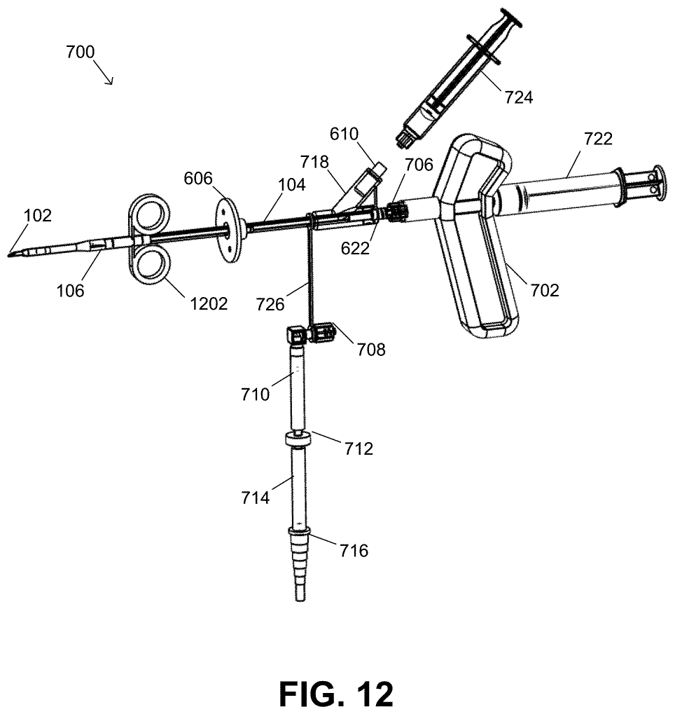

Referring now to FIG. 1, an exemplary variation of the rapid deployment chest port 100 in a contracted state is shown. The rapid deployment chest port 100 may include one or more blades 102, a frame 104, one or more internal expanding flanges 106, a check valve 108, an internal expansion mechanism 110, one or more external expanding flanges 120, and/or a dial mechanism 122. In some variations a blade 102 includes a sharp protrusion at a bottom of the rapid deployment chest port 100. The blade 102 includes a sharpened surface and non-limiting examples of the blade include a knife, a needle, a scalpel, a double-bladed scalpel, or other object with a surface of sufficient sharpness to penetrate through the thorax into the pleural space. In exemplary variations, the blade 102 is pointed, allowing the desired blunt dissection with minimal effect on the exterior of the patient's body. In some variations, the blade 102 may be blunt, such as in a cone shape, as seen in FIGS. 7 and 12. In some variations, the blade 102 may be angled or curved, such as the point of a fountain pen, to naturally guide the blade over the intended rib, as seen in FIG. 12. The blade may be realized with or without an internal lumen. In variations including the lumen, the lumen may be used in conjunction with a syringe or other air-tight device to produce a vacuum while the device is advanced through the patient's tissue. For example, the blade may be fluidly connected to the frame and/or handle. The blade is attached to the distal end of the rapid deployment chest port such that it may penetrate through the patient to the pleural space without the need for a separate scalpel. This allows for a more precise incision that is sized for the rapid deployment chest port without creating a wider than necessary opening, which is often the case with a scalpel.

The blade 102 may be connected or connectable to a frame 104. Frame 104 may be comprised of any suitable material, such as plastic or steel. In other variations, the frame 104 may be substantially cylindrical. In additional examples, the frame may further include a peel away introducer at its distal end. In some specific examples, a frame 104 may be roughly pentagonal in shape, with one or more appendages extending from the point of the pentagon.

Other shapes are within the scope of the invention. A non-limiting example of these one or more appendages is shown as the frame appendages 104A and 104B shown in the exemplary variation of FIG. 1. In such a variation, the blade 102 may be attached to only one of those frame appendages. By way of non-limiting example, FIG. 1 depicts the blade 102 attached to right frame appendage 104B, but the blade 102 could also be attached to the left frame appendage 104A. The frame 104 is surrounded on the bottom by the blade 102, on each side by an internal expansion mechanism 110, and may contain within it a check valve 108. One or more internal expanding flanges 106 may also be attached to the frame 104 at a point near the blade 102. Additional variations may include a blade that is suitably sized and shaped to receive a frame following insertion.

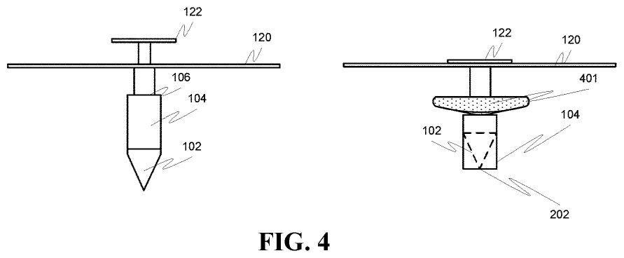

Internal expanding flanges 106 are connected to the frame 104 near the blade 102. In some variations, the internal expanding flanges 106 may additionally include a sleeve, such as a mesh net. In other variations, the internal expanding flanges 106 include a balloon. The balloon may expand inside the pleural space to secure the rapid deployment chest port in place. In some variations, the internal expanding flanges 106 are made of a sufficiently rigid material to allow them to operate to force open a larger area within the pleural space. Similarly, external expanding flanges 120 may be made of the same rigid material. In other variations, the internal expanding flanges and the external expanding flanges may be made of a highly compliant material such that they may be operable to conform to the body and minimize damage to the body. The external expanding flanges 120 are located on the opposite end of the rapid deployment chest port 100 relative to the blade 102. The external expanding flanges serve to stop the downward movement of the rapid deployment chest port into a patient and thus, in some variations, rest on a patient's body upon insertion of the rapid deployment chest port 100. In some variations, the external expanding flanges 120 do not expand when the dial mechanism 122 is engaged. One or both of the internal expanding flanges 106 and external expanding flanges 120 may serve to secure the rapid deployment chest port 100 in place on the patient's body. As displayed in FIG. 4, in some variations, the external expanding flange 120 may be in the form of a disk or a plate. In some variations, the external expanding flange may be slidable along a length of the frame. In at least some variations, the external expanding flange may be locked or secured in place once resting on the patient's body. In some variations, the plate may be padded. In other variations, one or both of internal expanding flanges 106 and external expanding flanges 120 may comprise a stationary (fixed) balloon, an adjustable balloon (which adjustment may be achieved using dial mechanism 122 or a syringe through an external valve port), a stationary pad, or an adjustable pad. The internal expanding flanges and the external expanding flanges may be used in combination, on either side of the incision, to secure the rapid deployment chest port to the patient in the proper location.

Running throughout the frame 104 may be an internal expansion mechanism 110. The internal expansion mechanism 110 connects a dial mechanism 122, which may be located at the top of the rapid deployment chest port 100, to the internal and external expanding flanges 106 and 120, respectively. By way of non-limiting example, the internal expansion mechanism may comprise one or more of: a spring, chemical reaction, or a wheel. The dial mechanism 122 controls the expansion and contraction of the internal expansion mechanism 110. In exemplary variations, the dial mechanism comprises a rotary element; however, any means for engaging, expanding, and contracting the internal expansion mechanism 110 is contemplated herein. By way of non-limiting examples of non-rotary dial mechanism variants, the dial mechanism may comprise a plunger, a button, a switch, a slide-ratcheting mechanism, or a digital controller capable of interfacing with a user by one or more of: Bluetooth, NFC, Wi-Fi, 3G, LTE, or touch screen. The dial mechanism may comprise a finite number of pre-determined settings, using a plurality of stops or a pawl, or it may adjust continuously. The dial mechanism 122 may assist in securing the rapid deployment chest port 100 in place,

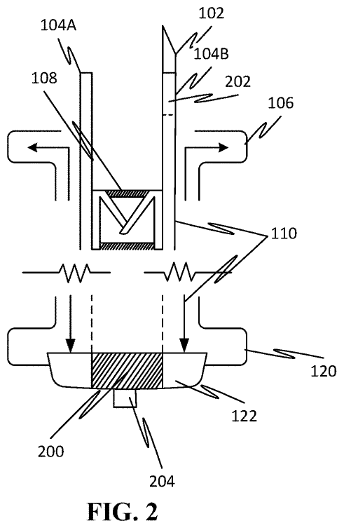

Referring now to FIG. 2, an expanded position of the rapid deployment chest port 100 is shown. In some variations, when turned, the dial mechanism 122 causes the expanding flanges 106 and 120 to expand by way of the internal expansion mechanism 110. In some variations, the dial mechanism 122 may lock in place upon reaching the desired expansion setting. In variations in which the frame 104 comprises frame appendages 104A and 104B, when the rapid deployment chest port 100 is in an expanded state, the frame appendages 104A and 104B are pulled apart from each other, along with the blade 102.

Upon insertion, the rapid deployment chest port 100 may create an airtight seal between the pleural space and the exterior of the patient's body. In some variations, the external expanding flange 120 comprises a flexible material that molds to the shape of the patient's body.

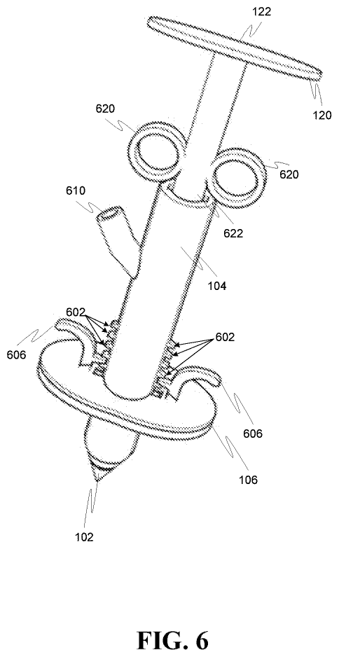

Upon expansion, then, air or fluid can only escape through the air escape opening 200. The air escape opening 200 divides the frame 104 horizontally and allows trapped air to escape from the patient's thorax. In exemplary variations, when the rapid deployment chest port 100 is in an expanded state, an embedded check valve 108 is exposed. The check valve 108 may serve as a one-way valve for air moving throughout the air escape opening 200. The check valve 108 allows the air trapped inside the thorax to drain through the air escape opening 200 and out of the patient's body, while not permitting any additional air in. In other variations, the check valve 108 allows fluid trapped in the pleural space to be removed through the air escape opening 200 or a lumen in the frame and out of the patient's body. For example, increased pressure within the pleural space may cause air or fluid to move through the frame without use of a suction source. In other examples, the check valve may be connected to a suction source to further assist in air or fluid removal. In some variations, the check valve 108 comprises a passive, flexible, one-way valve such as a Heimlich valve. In some variations, the check valve 108 may be placed exterior to the rapid deployment chest port, as shown in FIG. 6. In some variations, two or more check valves 108 may be used. These check valves 108 may be interior to the rapid deployment chest port 100, embedded within the rapid deployment chest port 100, or exterior to the rapid deployment chest port 100.

In some variations, a universal suction tube adapter 204 may be attached to the proximal end of the air escape opening 200. The universal suction tube adapter 204 may be adjusted to various industry-standard sizes, such as 8 French, 16 French, 20 French, 24 French, 28 French, 36 French, and 40 French, depending on the needs of the situation and the chest tubes available. In some variations, the universal suction tube adapter may include a male or female luer connector. Thus, a doctor or other medical attendant may feed a chest tube into the patient's thorax cavity, in accordance with the current medical treatment for tension pneumothorax, and continue the safe removal of the trapped air or fluid.

In some variations, when the rapid deployment chest port 100 is in an expanded state, the blade 102 may retract into the frame 104 at a blade retraction slot 202. The blade retraction slot 202 may be any means for ensuring that the point of the blade 102 is not exposed to the inside of the patient's body after retraction. In some variations, the blade retraction slot 202 may comprise a means for wiping the tip of the blade 102. Such means may include, for example, a narrow slot opening or an absorbent membrane. In some variations, the blade 102 is connected to the dial mechanism 122; thus, the retraction occurs due to the engaging of the dial mechanism 122. For example, if the dial mechanism 122 comprises a dial with pre-determined stops, the blade 102 may retract slowly as the dial mechanism 122 is turned. In other variations, such as where the dial mechanism 122 comprises a plunger or other binary engagement mechanism, the blade 102 may retract instantly upon engaging the dial mechanism 122.

In some variations, such as an variation in which the dial mechanism 122 is a plunger, activation of the dial mechanism 122 may cause several simultaneous reactions in the rapid deployment chest port 100. By way of non-limiting example, activating a plunger may do one or more of: extend the blade from the distal end of the frame; expand the external expanding flange 120; expand the internal expanding flange 106; retract the blade 102 into the blade retraction slot 202; deploy a sleeve from the internal expanding flange 106.

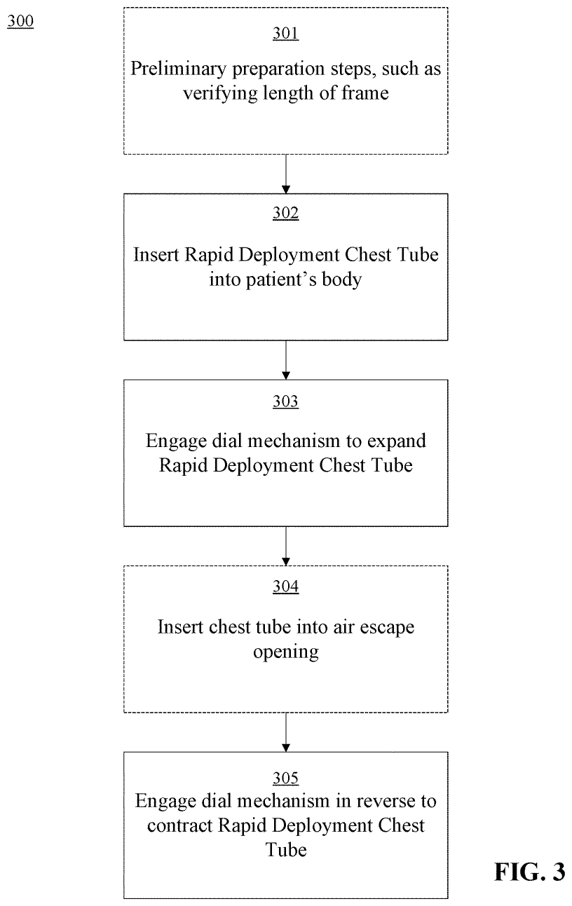

Referring now to FIG. 3, an exemplary variation of a method for using a rapid deployment chest port, such as treating tension pneumothorax 300 is shown. At optional step 301, preliminary steps are taken to prepare for the insertion of the rapid deployment chest port 100. These steps may comprise adjusting the length of the frame 104, using the internal expansion mechanism 110 to ensure an appropriate insertion depth, or preparing a patient's body. In exemplary variations, the depth of insertion is a function of the distance between the blade 102, which leads the insertion into the patient's body, and the external expanding flange 120, which stops the insertion when it comes to rest against the patient's body. Additionally, depending upon the patient, too shallow an insertion may be ineffective; too deep an insertion may be fatal. Thus, this preliminary calibration is crucial. In some variations, the method may further include using a syringe connected to the plunger to aspirate a small volume from the pleural space to confirm the rapid deployment chest port is inserted to the correct depth. The method may further include adjusting the depth of the rapid deployment chest port, if necessary. Other preliminary steps, such as sanitizing the blade, may be required in some variations or situations; however, in exemplary variations, the rapid deployment chest port 100 is stored in sterile, self-contained packaging designed for rapid deployment, and the rapid deployment chest port itself may be coated in one or more of: a disinfectant, antiseptic fluid, or anesthetic. Accordingly, in exemplary variations, optional step 301 will be minimal, if present at all.

At step 302, the rapid deployment chest port 100 is inserted into the patient's body. Due to the durability of the thoracic cavity, this insertion may require considerable force. In some variations, it may be desirable to access the pleural space indirectly, such as through the patient's axilla. In exemplary variations, the insertion is complete when the external expanding flange 120 rests against the patient's body.

At step 303, the dial mechanism 122 is engaged. In some variations, a sleeve around the internal expanding flange 106 will deploy when the rapid deployment chest port 100 is in an expanded state. In other variations, the internal expanding flange is a balloon that is deployed by filling it with air. In some variations, such as gradual engagement variations as when the dial mechanism 122 comprises a dial with pre-determined stops, one or more of the following expansion/contraction steps may occur gradually: (a) expand or slide the external expanding flange 120; (b) expand the internal expanding flange 106; (c) retract the blade 102 into the blade retraction slot 202 or into the lumen of the frame. In other variations, such as binary or instant engagement variations as when the dial mechanism 122 comprises a plunger, the aforementioned expansion/contraction steps may occur instantly or with minimal delay or discontinuities. Regardless, at the conclusion of step 302, the rapid deployment chest port 100 will be in at least a partially expanded state.

At optional step 304, air or fluid may exit through the air escape opening 200 via, in some variations, the universal suction tube adapter 204. The chest tube allows the tension pneumothorax treatment to proceed according to the current and known methods. In some examples, the plunger may be removed and a check valve, by way of a Leur connector, may be connected to a 1-way valve at the proximal end of the frame. In this example, a stepped connector connected to the proximal end of the check valve may be connected to a suction source to remove air or fluid from the pleural space.

Finally, at step 305, the dial mechanism 122 is engaged in reverse to contract the rapid deployment chest port 100. If the sleeve was deployed at step 303, it may wrap around one or more of: the internal expanding flange 106; the blade 102; the blade retraction port 202; the check valve 108; or the frame 102. When the internal expanding flange is a balloon, the method may further include deflating the balloon prior to removal of the rapid deployment chest port. The rapid deployment chest port 100 may then be safely removed from the patient's body.

Referring now to FIG. 4, alternative views of an alternative variation of the rapid deployment chest port 100 are shown. Notably, in this variation, the blade 102 rests over the entire frame; thus, there are no frame appendages 104A and 104B. Instead, once the dial mechanism 122 is engaged, in some variations the blade 102 retracts, and one or more of the expanding flanges expand, but the frame may not be transformed. FIG. 4 also demonstrates a variation in which the dial mechanism 122 comprises a plunger, and the internal expanding flange 106 deploys a sleeve 401 upon the plunger being pressed. In this exemplary, non-limiting variation, the external expanding flange 120 is already at its expanded size prior to the dial mechanism 122 being engaged.

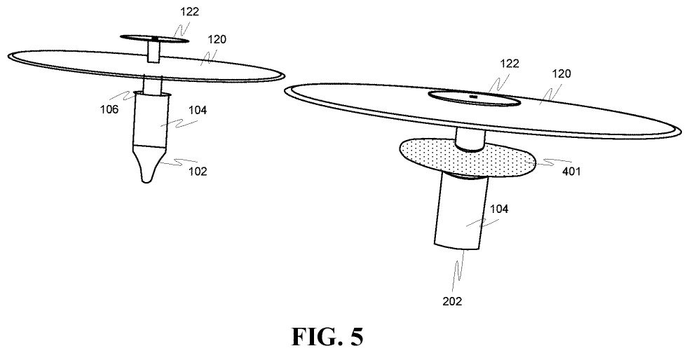

Referring now to FIG. 5, alternative, angular views of the alternative variation of FIG. 4 are shown.

Referring now to FIG. 6, another alternative variation is shown. In this variation, the dial mechanism 122 comprises a plunger. It will be recognized by those skilled in the art that the plunger can be independent of a dial mechanism. In some variations, then, the dial mechanism 122 and the external expanding flange 120 may serve the same function. In some variations, finger grips 620 may be present to assist the user in guiding the rapid deployment chest port 100 to the desired spot. In some variations, the plunger structure may be removable from the frame 104. The plunger may be inserted into the frame 104 through plunger port 622. In some variations, plunger port 622 comprises grooves. In some variations, external valve port 610 may feed into frame 104. As described above, external valve port 610 may operate to allow a check valve to be inserted into frame 104 without the check valve needing to be integrated into rapid deployment chest port 100.

Additionally, internal expanding flange 106 may have one or more groves or extrusions 602 to secure the frame 104 to an insertion stabilization platform 606. The insertion stabilization platform 606 may be integrated into the rapid deployment chest port 100 or be a separate piece through which the frame 104 and blade 102 can be inserted. The insertion stabilization platform 606 may assist in securing the rapid deployment chest port in place. The insertion stabilization platform 606 may comprise a balloon or pad, which balloon or pad may be stationary or adjustable. In some variations, the insertion stabilization platform 606 is adjustable by way of the extrusions 602. In some variations, extrusions 602 may comprise a sliding-ratcheting mechanism. In some variations, the insertion stabilization platform may have a coating of anesthetic or an anti-septic compound.

In some variations, rapid deployment chest port 100, as illustrated, may be modular. By way of non-limiting example, the rapid deployment chest port 100 may comprise three distinct pieces: a dial mechanism 122 (comprising finger grips 620, and a shaft linking the dial mechanism 122 to the blade 102); a frame 104 (comprising, as shown, the external valve port 610 and, in some variations, extrusions 602); and the optional insertion stabilization platform 606. In some variations, blade 102 may already be secured to the frame 104 or the insertion stabilization platform 606. stylet

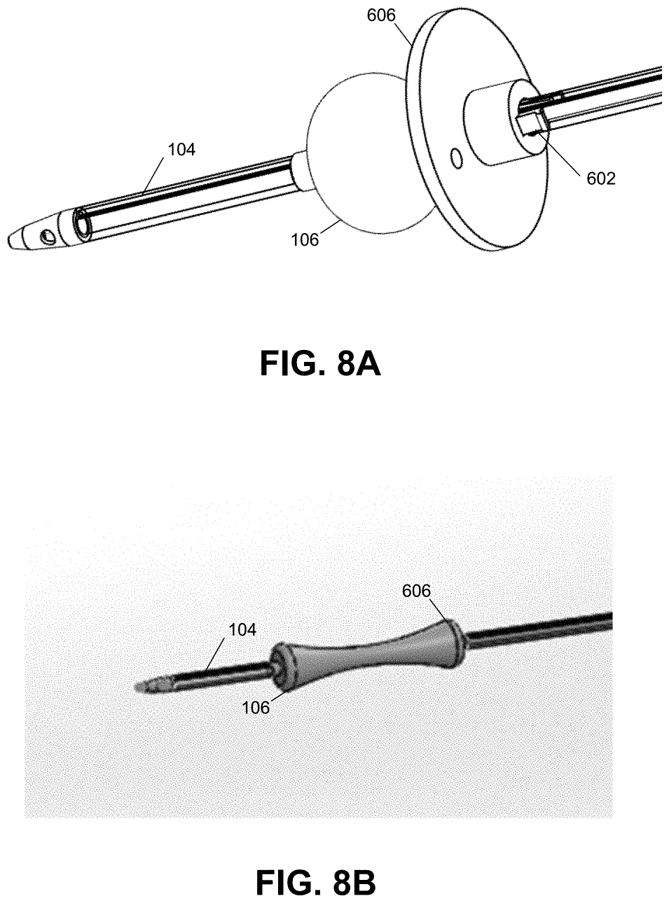

Referring now to FIGS. 7, 8A and 8B, another alternative variation is shown. In some variations, the rapid deployment chest port 700, as illustrated, may be modular. By way of non-limiting example, the rapid deployment chest port 700 may include a plunger 704 linking the handle 702 (e.g., finger grips) to the blade 102, a frame 104 (comprising a lumen for the plunger 704 and air and/or fluid, a Y-hub with an external valve port 610 and, in some variations, a plunger port 622 with a luer connector at the proximal end); and a stabilization component. In some variations, the stabilization component may include an internal expanding flange 106 (in this instance, comprising a balloon) and an external expanding flange (in this instance, comprising an insertion stabilization platform 606). In a variation, the plunger 704 may be a stylet shaft extending the length of the frame. In some variations, blade 102 may be secured to the frame 104 or the distal end of the plunger.

In some variations, the frame 104 may be compliant, such that it may be compressed. In additional variations, the frame 104 may be a catheter, such as a silicone catheter, or a thermo plastic or rubber extrusion. In some variations, the frame may include a lip to aid in the insertion of the rapid deployment chest port, such that the frame does not collapse during insertion. In other variations, the frame may not include a lip when a peel away introducer is used to reinforce the frame during insertion. In addition, the use of an introducer may compress the outer diameter of the frame at the site of insertion. Thus, in some examples, the diameter of the frame may depend on the use of an introducer. In a variation, the frame may have a diameter ranging from 5 French to 40 French. In some variations, the frame may have a diameter of 5 French. In some variations, the frame may have a diameter of 8 French. In some variations, the frame may have a diameter of 10 French. In some variations, the frame may have a diameter of 16 French. In some variations, the frame may have a diameter of 20 French. In some variations, the frame may have a diameter of 25 French. In some variations, the frame may have a diameter of 30 French. In some variations, the frame may have a diameter of 35 French. In some variations, the frame may have a diameter of 40 French.

In this variation, plunger 704 is connected to the blade 102 at the distal end and connected to finger grips at the proximal end. In other variations, the blade may be integrated with the plunger, such that they are a single element. For example, the plunger may have a tapered distal end, forming a blade. In other examples, the plunger may terminate in a blade. In some variations, the blade 102 may be any structure capable of piercing the skin and penetrating through the body to the pleural space. Non-limiting examples of blades include a needle, sharp tip, and/or or knife edge. In at least one example, the blade 102 may be a sharp silicone tip. In some variations, the finger grips may be a handle 702. The handle 702 may have an upper and lower portion, and the lower portion may be longer than the upper portion. In some variations, the upper and lower portions may be angled to provide an ergonomic handle. In some variations, the handle may be present to assist the user in guiding the rapid deployment chest port 700 to the desired spot. In a variation, the handle may further include a syringe port 720 at the proximal end of the handle. In an example, an aspiration syringe 722 may be attached to the syringe port 720 for testing the placement of the rapid deployment chest port 700. In this example, a user may withdraw the aspiration syringe 722 to identify the fluid or air located at the blade/distal end of the frame. If the rapid deployment chest port 700 is in the incorrect location, the user may adjust the placement by moving the handle towards or away from the patient, as appropriate. The aspiration syringe may again be used to test the placement of the rapid deployment chest port 700. The aspiration syringe 722 may be removed from the syringe port 720 on the handle once the correct placement of the rapid deployment chest port 700 is confirmed.

In some variations, the plunger structure may be removable from the frame 104. The plunger 704 may be inserted into the frame 104 through a plunger port 622 on the frame 104. In some variations, the plunger port 622 may be on a Y-hub 718. The plunger 704 may pass through a lumen in the frame and end in the blade 102. In some variations, the plunger port 622 may include a luer connector. In some variations, the handle may include a reciprocal luer connector 706 to connect the handle to the plunger port 622. The plunger 704 may be already inserted, and then removed. For example, the plunger 704 may be removed from the frame 104 when the rapid deployment chest port 700 is placed in the pleural space of the patient. In some variations, when the plunger 704 is removed, a reciprocal luer connector 708 may connect to the plunger port 622 to attach an external check valve assembly to the frame 104. The external check valve assembly may operate to allow a check valve to be inserted into frame 104 without the check valve needing to be integrated into rapid deployment chest port 700. In some variations, the check valve assembly may include the luer connector 708, a check valve 712, valve outlet tubing 710 connected to the luer connector and distal to the check valve, valve inlet tubing 714 proximal to the check valve, and a connector 716 to a suction source. In some variations, the connector may include a stepped connector. The stepped connector may attach to a suction source, such that fluid and/or air trapped in the pleural space may be pulled through the frame 104 and to the suction source. In some variations, when not in use or connected to the plunger port 622, the check valve assembly may be attached to the frame 104 by a strap 726 so that it may then me readily available when needed to connect to the plunger port 622.

The frame 104 may further include an external valve port 610 on the Y-hub 718. In some variations, the external valve port 610 may be a luer activated valve. The luer activated valve may be connected to a lumen in the frame 104, which may then be connected to the internal expanding flange. In some examples, a syringe 724 may connect to the luer activated valve to supply air to the internal expanding flange when the internal expanding flange is a balloon.

In some variations, the rapid deployment chest port includes a stabilizing component configured to stabilize the frame inside and outside the chest cavity of a patient. The stabilizing component may be a single component configured to expand in the interior and exterior of the chest cavity of the patient. In some variations, the single stabilizing component is a balloon, as seen in FIG. 8A. In some examples, the stabilizing component includes an internal expanding flange attached to the outer diameter of the frame and external expanding flange (or an insertion stabilization platform) attached to the outer diameter of the frame proximal to the internal expanding flange.

In some variations, the external expanding flange is an insertion stabilization platform 606, as seen in FIG. 7. The insertion stabilization platform 606 may assist in securing the rapid deployment chest port in place. In a variation, the insertion stabilization platform may be a disc and may have a diameter sufficient to support and secure the rapid deployment chest port. In a variation, the insertion stabilization platform may have a diameter of 5 mm to 60 mm. In some variations, the insertion stabilization platform may have a diameter of at least 5 mm. In some variations, the insertion stabilization platform may have a diameter of at least 10 mm. In some variations, the insertion stabilization platform may have a diameter of at least 15 mm. In some variations, the insertion stabilization platform may have a diameter of at least 20 mm. In some variations, the insertion stabilization platform may have a diameter of at least 30 mm. In some variations, the insertion stabilization platform may have a diameter of at least 40 mm. In some variations, the insertion stabilization platform may have a diameter of at least 50 mm. In some variations, the insertion stabilization platform may have a diameter of less than 60 mm.

The insertion stabilization platform may be placed a distance from the blade and provide an external surface for securing the placement of the rapid deployment chest port 700. In some variations, the insertion stabilization platform 606 may rest on the patient when the rapid deployment chest port is inserted the proper distance. The location of the insertion stabilization platform may be adjustable. The insertion stabilization platform may be initially placed at a distance from the blade to mitigate the risk of injury to internal anatomy during insertion of the rapid deployment chest port. In some variations, the insertion stabilization platform may be located from 3 cm to 7 cm from the blade. Most patients' pleural spaces are within 6.5 cm from the surface of the body, thus an initial spacing of the insertion stabilization platform of 6.5 cm may allow the rapid deployment chest port to clear the thickness of most patients while limiting insertion depth to prevent internal injury. In a variation, the insertion stabilization platform 606 may not be adjustable beyond 7 cm from the blade 102.

In a variation, the insertion stabilization platform 606 may have one or more groves or extrusions 602, such as a fixation flexure, to secure the insertion stabilization platform 606 to the frame. The insertion stabilization platform 606 may be integrated into the rapid deployment chest port 700 or be a separate piece through which the frame 104 and blade 102 can be inserted.

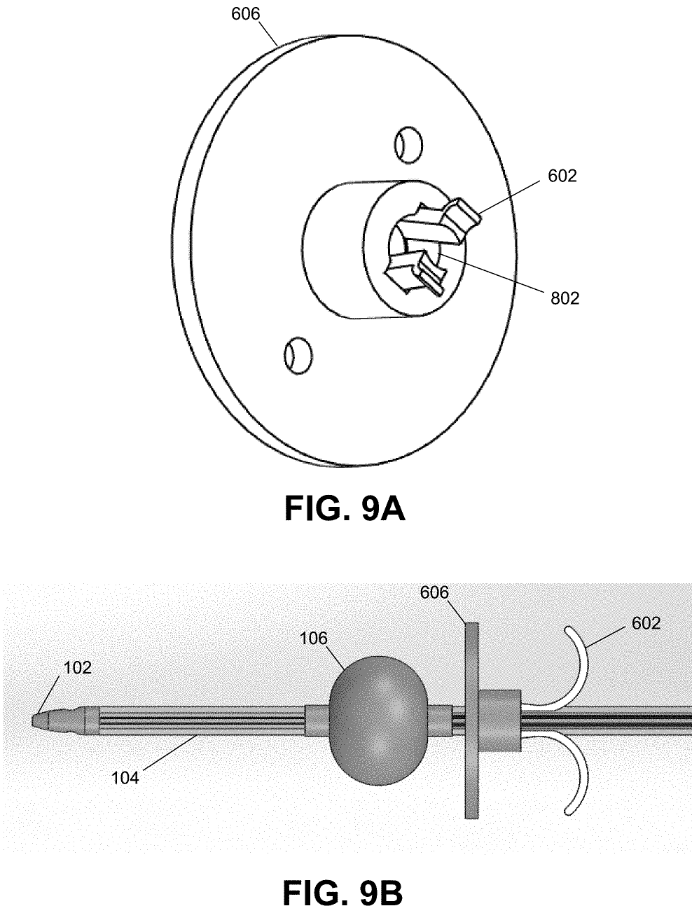

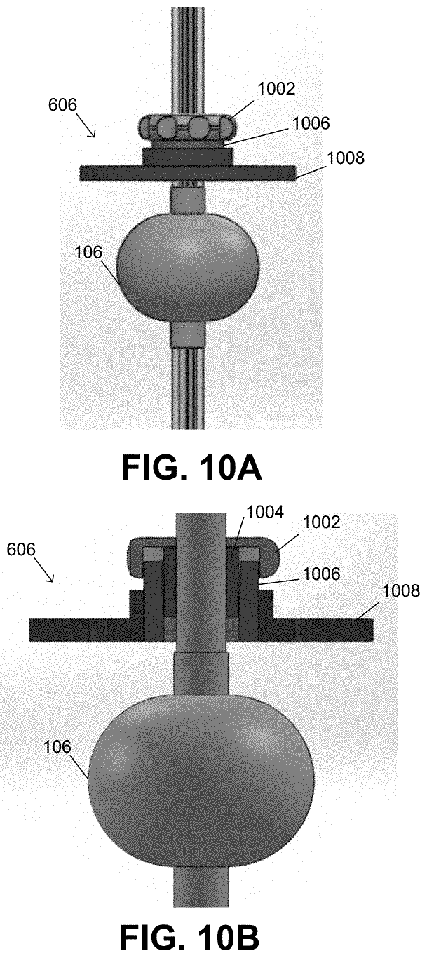

In some variations, the insertion stabilization platform 606 is adjustable by way of the fixation flexure, as seen in FIGS. 9A, 9B, 10A, and 10B. In some examples, the insertion stabilization platform may include an opening 802 to allow the insertion stabilization platform to be slid along the frame 104 and then secured into place against the patient using the fixation flexure after the rapid deployment chest port has been inserted the proper distance. The fixation flexure may have varying lengths and shapes to allow for ease of gripping and sliding the insertion stabilization platform. The fixation flexure may have two extensions, each with an outward extending flange, as seen in FIGS. 9A and 9B. In some examples, the two extensions and outward extending flanges may be extended and curved, as seen in FIG. 9B. In some variations, the fixation flexure may be pinched to allow the insertion stabilization platform to slide along the frame, and release of the fixation flexure secures the insertion stabilization platform in place. In other variations, the extrusions may include a sliding-ratcheting mechanism for moving and securing the insertion stabilization platform to the frame. In additional variations, the insertion stabilization platform 606 may form a seal around the frame to hold the insertion stabilization platform in place to limit initial insertion depth and prevent frame migration. For example, the insertion stabilization platform 606 may include a compression fitting, as seen in FIGS. 10A and 10B. In some variations, the compression fitting may include a knob 1002, compression sleeve 1004, compression hub 1006, and/or a compression pad 1008, as seen in FIGS. 10A and 10B. In some examples, the insertion stabilization platform 606 may include a threaded connection between the knob 1002 and compression hub 1006. As the knob 1002 is tightened down onto the hub 1006, the compression sleeve 1004 is compressed against the frame 104, preventing relative motion. In some variations, the insertion stabilization platform may include a balloon or pad on the patient facing surface, where the balloon or pad may be stationary or adjustable. In some variations, the insertion stabilization platform may have a coating of anesthetic or an anti-septic compound.

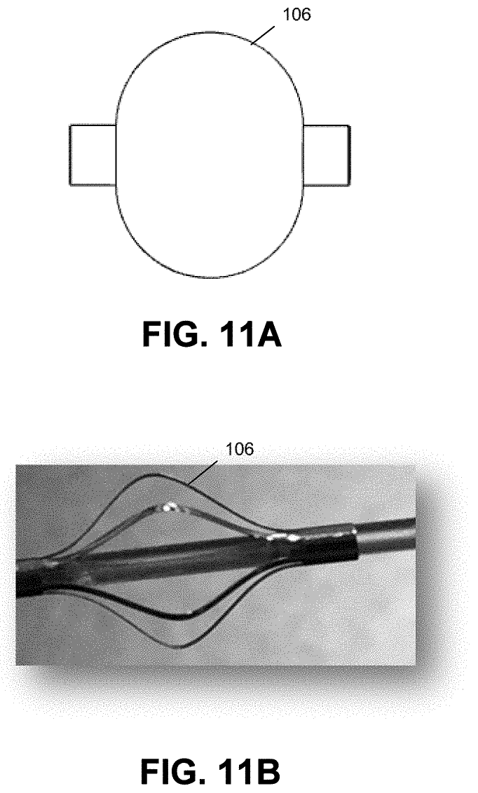

In some variations, the rapid deployment chest port may include an internal expanding flange 106 connected to the frame 104 near the blade 102. In a variation, the internal expanding flange 106 may be a balloon, as seen in FIGS. 7 and 11A. In some variations, the balloon is a compliant balloon. For example, the balloon may be a silicone balloon with a hardness of Shore A 50 or less. In other variations, the internal expanding flange may be any expandable structure made of a material suitable for creation of flexure elements, such as nitinol. In at least one variation, the internal expanding flange 106 may be an expandable nitinol ascot or a silicone covered expandable nitinol ascot, as seen in FIG. 11B.

The diameter of the internal expanding flange may range from about 5 mm to 55 mm. In some non-limiting variations, the internal expanding flange may have a diameter of at least 5 mm. In some non-limiting variations, the internal expanding flange may have a diameter of at least 21 mm. In some non-limiting variations, the internal expanding flange may have a diameter of at least 27 mm. In some non-limiting variations, the internal expanding flange may have a diameter of at least 38 mm. In some non-limiting variations, the internal expanding flange may have a diameter of at least 52 mm. In some non-limiting variations, the internal expanding flange may have a diameter of less than or equal to 55 mm. In some non-limiting variations, the internal expanding flange may have a diameter of less than or equal to 52 mm. In some non-limiting variations, the internal expanding flange may have a diameter of less than or equal to 38 mm. In some non-limiting variations, the internal expanding flange may have a diameter of less than or equal to 27 mm. In some non-limiting variations, the internal expanding flange may have a diameter of less than or equal to 21 mm. In some non-limiting variations, the internal expanding flange may have a diameter of less than or equal to 15 mm. In some non-limiting variations, the internal expanding flange may have a diameter of less than or equal to 10 mm.

The diameter of the insertion stabilization platform may be selected based on the diameter of the frame to limit damage to the patient during insertion and removal. The internal expanding may be large enough to provide sufficient force to prevent dislodgment or frame migration during the course of normal events is desirable while mitigating the risk that the rapid deployment chest port can damage tissue or otherwise harming the patient if the frame is exposed to uncommonly large forces. In at least some variations, the ratio between the diameter of the insertion stabilization platform and the diameter of the frame may range from 2.5 to 6. In one non-limiting variation, the ratio between the diameter of the insertion stabilization platform and the diameter of the frame is at least 2.5. In one non-limiting variation, the ratio between the diameter of the insertion stabilization platform and the diameter of the frame is at least 3.0. In one non-limiting variation, the ratio between the diameter of the insertion stabilization platform and the diameter of the frame is at least 4.0. In one non-limiting variation, the ratio between the diameter of the insertion stabilization platform and the diameter of the frame is at least 5.0. In one non-limiting variation, the ratio between the diameter of the insertion stabilization platform and the diameter of the frame is less than or equal to 6.0. In one non-limiting variation, the ratio between the diameter of the insertion stabilization platform and the diameter of the frame is less than or equal to 5.0. In one non-limiting variation, the ratio between the diameter of the insertion stabilization platform and the diameter of the frame is less than or equal to 4.0. In one non-limiting variation, the ratio between the diameter of the insertion stabilization platform and the diameter of the frame is less than or equal to 3.0. In at least one variation, the ratio between the diameter of the insertion stabilization platform and the diameter of the frame may range from 3 to 5. In an example, the ratio between the diameter of the insertion stabilization platform and the diameter of the frame may be 5.

In other non-limiting examples, the balloon may be made of Urethan, Pebax or any other thermoformed or extruded material. In a variation, the balloon may have a volume of 2 mL to 10 mL when used with a 16 French frame. The volume of the balloon, and thus the diameter of the balloon, may be adjusted based on the diameter of the frame based on the ratio of internal expanding flange to frame diameter. In a variation, the balloon may have a volume of 2 mL. In a variation, the balloon may have a volume of 3 mL. In a variation, the balloon may have a volume of 4 mL. In a variation, the balloon may have a volume of 5 mL. In a variation, the balloon may have a volume of 6 mL. In a variation, the balloon may have a volume of 7 mL. In a variation, the balloon may have a volume of 8 mL. In a variation, the balloon may have a volume of 9 mL. In a variation, the balloon may have a volume of 10 mL.

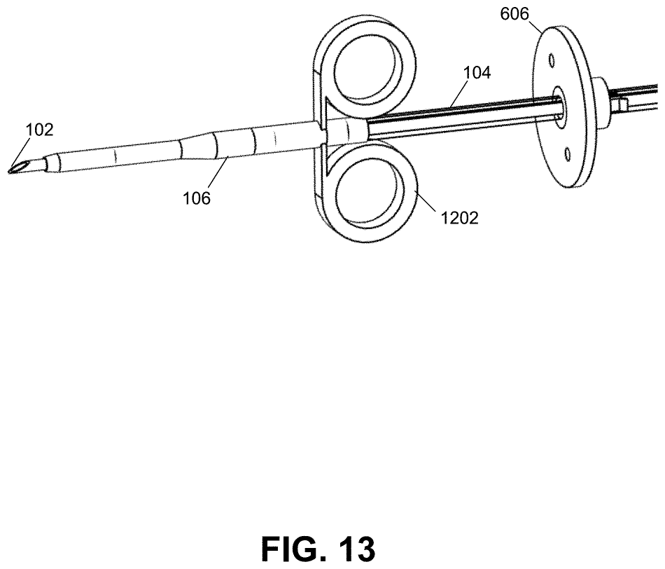

The external valve port 610 may be fluidly connected to the stabilizing component to expand and deflate the internal expanding flange and/or insertion stabilization platform. In a variation, when the internal expanding flange is a balloon, the external valve port 610 may be fluidly connected to the internal expanding flange to facilitate the connection of a syringe to expand and deflate the balloon with air. The internal expanding flange may initially be deflated and against the outer diameter of the frame to aid in insertion of the rapid deployment chest port. In FIGS. 12 and 13, the internal expanding flange 106 is shown in the deflated state against the frame 104.The internal expanding flange may then expand inside the pleural space to secure the rapid deployment chest port once it is properly in place. One or both of the internal expanding flange 106 and insertion stabilization platform 606 may serve to secure the rapid deployment chest port 700 in place on the patient's body.

The balloon and the insertion stabilization platform may be used in combination, on either side of the incision, to secure the rapid deployment chest port to the patient in the proper location. In some variations, the combination of the insertion stabilization platform and the internal expanding flange may allow the rapid deployment chest port to create an airtight seal between the inside and outside of the patient's body. This may allow for the efficient removal of air or fluid from the pleural space, reduce the risk of infection at the insertion site, and reduce the amount of time to treat the patient.

Referring now to FIG. 12, an alternative variation of the alternative variation of FIG. 7 is shown. In this variation, the frame 104 may further include a peel away introducer 1202 for assisting in the insertion of the rapid deployment chest port 700, as further seen in FIG. 13. In some variations, the stabilizing component may include a compressed expandable portion of the frame that is compressed during insertion and expands after insertion to contour around internal and external tissue at an insertion site to prevent frame migration following deployment. In at least one example, the frame is compressed by the peel away introducer and then expands within the incision once the introducer is removed. The peel away introducer 1202 includes a heat-shrink material that compresses the frame onto the plunger. At the distal end of the rapid deployment chest port the heat shrink may make a smooth transition from the plunger to the frame. In addition, the peel away introducer 1202 may include modeled finger grips that are used to peel the two halves of the heat-shrink material apart. In some variations, these finger grips may also be used to limit insertion depth. For example, the bottom of the finger grips may be used to mitigate the risk of injury to internal anatomy during insertion of the rapid deployment chest port by providing a stop to the insertion depth. In a variation, the distal end of the finger grips may be located from 3 cm to 7 cm from the blade. In this example, the location of the finger grips of the peel away introducer may allow the rapid deployment chest port to clear the thickness of most patients while limiting insertion depth to prevent internal injury. In a variation, the finger grips of the peel away introducer may not be located beyond 7 cm from the blade 102.

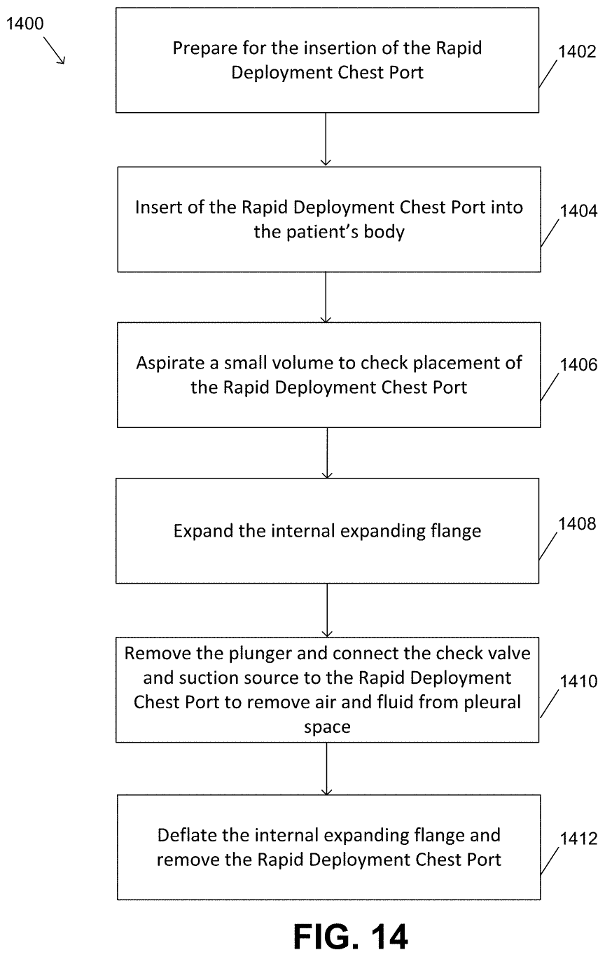

Referring now to FIG. 14, an exemplary variation of a method for accessing the pleural space of a patient 1400 is shown. The patient's pleural space may need to be accessed urgently or non-urgently. Non-limiting treatments or needs for accessing the pleural space include treatment of tension pneumothorax, treatment of non-tension pneumothorax, removal of fluid from trauma, drainage of a small amount of fluid, and/or administration medication to the pleural space. At optional step 1402, preliminary steps are taken to prepare for the insertion of the rapid deployment chest port. These steps may include adjusting the location of the insertion stabilization platform along the frame or preparing a patient's body. In exemplary variations, the depth of insertion is a function of the distance between the blade, which leads the insertion into the patient's body, and the insertion stabilization platform, which stops the insertion when it comes to rest against the patient's body. Additionally, depending upon the patient, too shallow an insertion may be ineffective; too deep an insertion may cause undue harm. Other preliminary steps, such as sanitizing the blade, may be required in some variations or situations; however, in exemplary variations, the rapid deployment chest port is stored in sterile, self-contained packaging designed for rapid deployment, and the rapid deployment chest port itself may be coated in one or more of: a disinfectant, antiseptic fluid, or anesthetic. Accordingly, in exemplary variations, optional step 1402 will be minimal, if present at all.

At step 1404, the rapid deployment chest port is inserted into the patient's body. In some variations, this may include inserting the blade of the plunger and distal portion of the frame of the rapid deployment chest port into the patient's chest cavity. Due to the durability of the thoracic cavity, this insertion may require considerable force. In some variations, it may be desirable to access the pleural space indirectly, such as through the patient's axilla. In exemplary variations, the insertion is complete when the insertion stabilization platform rests against the patient's body.

At optional step 1406, a syringe connected to the handle is used to aspirate a small volume from the pleural space to confirm the rapid deployment chest port is inserted to the correct depth. This step may further include adjusting the depth of the rapid deployment chest port, if necessary. Optional step 1406 may occur simultaneously with step 1404.

At step 1408, the stabilizing component is expanded in at least the inside of the patient's chest cavity. In some variations, the stabilizing component is the internal expanding flange. In some variations, the internal expanding flange is a balloon that is expanded by filling it with air through a syringe connected to the external valve port on the frame. In some variations, step 1408 may optionally include sliding or locking the insertion stabilization platform such that it rests on the patient's chest. At the conclusion of step 1408, the rapid deployment chest port may be securely set in the patient at the proper insertion depth for the patient.

At step 1410, plunger 704 may be removed from the frame and a check valve, by way of a luer connector, may be connected to a 1-way valve at the proximal end of the frame. In this example, a stepped connector connected to the proximal end of the check valve may be connected to a suction source to remove air or fluid from the pleural space.

Finally, at step 1412, the stabilization component, such as the internal expanding flange, is deflated prior to removal of the rapid deployment chest port. In some examples, this may include withdrawing air from the balloon using the syringe attached to the external valve port. The rapid deployment chest port may then be safely removed from the patient's body.

A number of embodiments of the present disclosure have been described. While this specification contains many specific implementation details, there should not be construed as limitations on the scope of any disclosures or of what may be claimed, but rather as descriptions of features specific to particular embodiments of the present disclosure. While embodiments of the present disclosure are described herein by way of example using several illustrative drawings, those skilled in the art will recognize the present disclosure is not limited to the embodiments or drawings described. It should be understood the drawings and the detailed description thereto are not intended to limit the present disclosure to the form disclosed, but to the contrary, the present disclosure is to cover all modification, equivalents and alternatives falling within the spirit and scope of embodiments of the present disclosure as defined by the appended claims.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted the terms “comprising”, “including”, and “having” can be used interchangeably.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while method steps may be depicted in the drawings in a particular order, this should not be understood as requiring that such operations be performed in the particular order shown or in a sequential order, or that all illustrated operations be performed, to achieve desirable results.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. Nevertheless, it will be understood that various modifications may be made without departing from the spirit and scope of the claimed disclosure.

1. A rapid deployment chest port, comprising:
a frame comprising a lumen and a plunger port;
a removable plunger comprising a blade at the distal end, wherein the removable plunger is within the lumen of the frame; and
a stabilizing component configured to stabilize the frame inside and outside a chest cavity of a patient, the stabilizing component comprising:
a balloon attached to an outer diameter of the frame; and
an insertion stabilization platform attached to and slidable along the outer diameter of the frame proximal to the balloon.

2. (canceled)

3. The rapid deployment chest port of claim 1, further comprising an external valve port attached to the outer diameter of the frame.

4. The rapid deployment chest port of claim 3, wherein the external valve port is fluidly connected to the balloon.

5. (canceled)

6. (canceled)

7. (canceled)

8. The rapid deployment chest port of claim 1, wherein a compressed expandable portion of the frame is compressed by a peel away introducer.

9. The rapid deployment chest port of claim 8, wherein the peel away introducer comprises finger grips, wherein a bottom of the finger grips is no more than 7 cm from the blade.

10. (canceled)

11. (canceled)

12. (canceled)

13. The rapid deployment chest port of claim 1, wherein the insertion stabilization platform further comprises a fixation flexure operable to allow sliding of the insertion stabilization platform along the outer diameter of the frame and then secure the insertion stabilization platform to the frame.

14. The rapid deployment chest port of claim 1, wherein the insertion stabilization platform is no more than 7 cm away from the blade when the rapid deployment chest port is inserted into the chest cavity of the patient.

15. (canceled)

16. (canceled)

17. The rapid deployment chest port of claim 1, further comprising a handle with a connector operable to removably attach the handle to the plunger port at a proximal end of the frame.

18. The rapid deployment chest port of claim 17, wherein the handle further comprises a syringe port operable to receive an aspiration syringe.

19. The rapid deployment chest port of claim 17, wherein the plunger port is a 1-way valve.

20. The rapid deployment chest port of claim 1, external valve port is a 1-way valve operable to receive a syringe to expand the balloon.

21. The rapid deployment chest port of claim 1, wherein a ratio of a diameter of the balloon to a diameter of the frame is 2.5 to 6.

22. The rapid deployment chest port of claim 1, wherein the blade has a cone shape.

23. The rapid deployment chest port of claim 1, wherein the blade is a needle.

24. The rapid deployment chest port of claim 1, further comprising an external check valve assembly operable to connect to the plunger port and a suction source.

25. A method of removing air or fluid or both contained within a pleural space of a mammalian patient, the method comprising:
inserting the blade of the removable plunger and a distal portion of the frame of the rapid deployment chest port of claim 1 into a chest cavity of a patient;
expanding the balloon on the inside of the chest cavity of the patient; and
removing the plunger from the plunger port.

26. (canceled)

27. The method of claim 25, further comprising:
connecting a check valve and a suction source to the plunger port; and
removing air or fluid or both from the pleural space.

28. The method of claim 27, further comprising
deflating the balloon; and
removing the rapid deployment chest port from the patient.

29. (canceled)

30. The method of claim 25, further comprising aspirating a volume of air or fluid or both using a syringe attached to a handle of the rapid deployment chest port to confirm placement of the rapid deployment chest port within the pleural space of the patient.

31. The rapid deployment chest port of claim 24, wherein the external check valve assembly comprises:
a connector configured to connect to the plunger port,
a valve outlet tubing operably associated with the connector,
a check valve operably associated with the valve outlet tubing, and
a valve inlet tubing operably connected to the check valve and proximal to the check valve.
